(19) United States
(12) Patent Application Publication
(10) Pub. No.: US 2006/0183117 A1
(43) Pub. Date: Aug. 17, 2006

(54) Screening for alzheimer's disease

(76) Inventors: Margaret A Pericak-Vance, Chapel Hill, NC (US); Jeffery M Vance, Chapel Hill, NC (US); Johathan L Haines, Brentwood, TN (US)

Correspondence Address: MYERS BIGEL SIBLEY & SAJOVEC, PO BOX 37428, RALEIGH, NC, 27627, US

(21) Appl. No.: 10/520,779

(22) PCT Filed: Jul. 8, 2003
(86) PCT No.: PCT/US03/22259

§ 371 Date: Oct. 13, 2005

Related U.S. Application Data
(60) Provisional application No. 60/394,443, filed on Jul. 8, 2002.

(51) Int. Cl. C12Q 1/68

(52) U.S. Cl. 435/6

(57) ABSTRACT

The present invention discloses methods of screening a subject for Alzheimer's disease comprising detecting the presence or absence of a marker or functional polymorphism associated with a gene linked to Alzheimer's disease.

### CROSS REFERENCE TO RELATED APPLICATION

The present application claims priority to U.S. Provisional Application No. 60/394,443, filed Jul. 8, 2002, the disclosure of which is incorporated herein by reference in its entirety.

### FIELD OF THE INVENTION

This invention concerns methods of screening for neurological disorders by the screening of genetic risk factors. In particular, the present invention concerns Alzheimer's disease.

### BACKGROUND OF THE INVENTION

Degenerative diseases of the central nervous system included a number of neurodegenerative disease. Genetic studies of common complex neurodegenerative diseases, such as Alzheimer's disease, have focused on the identification of risk genes as targets for development of new treatments and improved diagnoses. This approach has identified the amyloid precursor protein (APP) (Goate et al., Nature 349:704-706 (1991)), presenilin 1 (PS1) (Sherrington et al., Nature 375:754-760 (1995)), presenilin 2 (PS2) (Levy-Lahad et al., Science 269:973-977 (1995); Rogaev et al., Nature 376:775-778 (1995)), and apolipoprotein E (APOE) (Corder et al., Science 261:921-923 (1993)) genes as contributing to risk in Alzheimer's disease. APP, PS1, and PS2 cause rare early-onset autosomal dominant Alzheimer's disease (5% of Alzheimer's disease cases), whereas APOE is associated with both risk and age at onset (AAO) in late-onset familial Alzheimer's disease, as well as in late- and early-onset sporadic Alzheimer's disease. Genomic screens in Alzheimer's disease (Kehoe et al., Hum Mol Genet 8:237-245 (1999); Pericak-Vance et al., Exp Gerontol 35:1343-1352 (2000) have recently localized additional but, as yet, unknown risk genes.

Alzheimer's disease is a progressive neurodegenerative disorder which is the predominant cause of dementia in people over 65 years of age. Clinical symptoms of the disease generally begin with subtle short term memory problems and as the disease progresses, difficulties with memory, language and orientation occur more frequently. In late stage Alzheimer's disease, ventricular enlargement and shrinkage of the brain may be observed by magnetic resonance imaging. Some characteristic changes in the Alzheimer's disease brain include neuronal loss in selected regions; intracellular neurofibrillary tangles (NFTs) in the neurons of the cerebral cortex and hippocampus; and neuritic plaques containing amyloids that may be further surrounded by dystrophic neuriteism reactive astrocytes and microglia. See, e.g., Wisniewski et al., Biochem. Biophys. Res. Comm. 192:359 (1993).

The NFTs characteristic of Alzheimer's disease consist of abnormal filaments bundled together in neuronal cell bodies. What are referred to as “Ghost” NFTs are also observed in Alzheimer's disease brains, presumably marking the location of dead neurons. Other neuropathical features of Alzheimer's disease include granulovacular changes, neural loss, gliosis and the variable presence of Lewy bodies. The identification between genetic loci and neurodegenerative changes or associations between genetic loci and the risk of developing a neurodegenerative disease may be useful in methods of diagnosing, screening and prognosing patients. They may also be used in therapeutic development methods.

Identification of further genes would open new avenues of research with the potential to delay onset beyond the natural life span. Present knowledge about genes contributing to AAO in neurodegenerative diseases clearly lags behind the understanding of genes contributing to risk. Recently, there has been growing interest in using AAO information as a quantitative trait, to identify genes that influence onset of disease (Daw et al., Am J Hum Genet 64:839-851 (1999), Daw et al., Am J Hum Genet 66:196-204 (2000); Duggirala et al. Am J Hum Genet 64:1127-1140 (1999)). Rapid development of methods of mapping quantitative trait loci (QTLs) for general pedigrees (Goldgar, Am J Hum Genet 47:957-967 (1990); Amos, Am J Hum Genet 54:535-543 (1994); Blangero et al. Genet Epidemiol 14:959-964 (1997)) has now made the search for novel genes affecting AAO feasible. Nevertheless, the genetic basis for Alzheimer's disease is not well understood, and there is a continued need to develop new genetic linkages and markers as well as identifying new functional polymorphisms that are associated with Alzheimer's disease. It may be advantageous to locate a gene or genes common to the disorder.

### SUMMARY OF THE INVENTION

The present invention demonstrates the identification of risk genes associated with Alzheimer's disease. These at risk linkage regions also indicate that LRP1 is a candidate gene for Alzheimer's disease. The present invention further discloses methods of screening a subject for Alzheimer's disease. The method comprises the steps of: detecting the presence or absence of a marker for Alzheimer's disease, or a functional polymorphism associated with a gene linked to Alzheimer's disease, with the presence of such a marker or functional polymorphism indicating that subject is afflicted with or at risk of developing Alzheimer's disease. The detecting step may include detecting whether the subject is heterozygous or homozygous for the marker and/or functional polymorphism, with subjects who are at least heterozygous for the functional polymorphism being at increased risk for Alzheimer's disease. The step of detecting the presence or absence of the marker or functional polymorphism may include the step of detecting the presence or absence of the marker or functional polymorphism in both chromosomes of the subject (i.e., detecting the presence or absence of one or two alleles containing the marker or functional polymorphism). More than one copy of a marker or functional polymorphism (i.e., subjects homozygous for the functional polymorphism) may indicate greater risk of Alzheimer's disease as compared to heterozygous subjects.

A further aspect of the present invention is the use of a means of detecting a marker, functional polymorphism or mutation as described herein in screening a subject for Alzheimer's disease as described herein.

The foregoing and other objects and aspects of the present invention are explained in detail in the drawings herein and the specification set forth below.

### BRIEF DESCRIPTION OF THE DRAWINGS

FIG. 1 is a chart depicting LRP1 single nucleotide polymorphisms on chromosome 12.

### DETAILED DESCRIPTION OF THE EMBODIMENTS

As noted above, the present invention provides a method of screening (e.g., diagnosing, detecting, determining or prognosing) for Alzheimer's disease in a subject. Subjects with which the present invention is concerned are primarily human subjects, including male and female subjects of any age or race.

The term “Alzheimer's disease” (AD) as used herein is intended to encompass all types of Alzheimer's disease, including sporadic and familial Alzheimer's disease, as well as late onset and early onset Alzheimer's disease.

The term “late-onset Alzheimer's disease” refers to Alzheimer's disease which has a time of onset after the subject reaches 40 years of age.

“Screening” as used herein refers to a procedure used to evaluate a subject for risk of Alzheimer's disease. It is not required that the screening procedure be free of false positives or false negatives, as long as the screening procedure is useful and beneficial in determining which of those individuals within a group or population of individuals are at increased risk of Alzheimer's disease. A screening procedure may be carried out for both prognostic and diagnostic purposes (i.e., prognostic methods and diagnostic methods).

“Prognostic method” refers to methods used to help predict, at least in part, the course of a disease. For example, a screening procedure may be carried out on a subject that has not previously been diagnosed with Alzheimer's disease or does not show substantial disease symptoms. The procedure allows one to obtain an indication of the future likelihood that the subject will be afflicted with Alzheimer's disease. In addition, a prognostic method may be carried out on a subject previously diagnosed with Alzheimer's disease when it is desired to gain greater insight into how the disease will progress for that particular subject (e.g., the likelihood that a particular patient will respond favorably to a particular drug treatment, or when it is desired to classify or separate Alzheimer's disease patients into distinct and different subpopulations for the purpose of conducting a clinical trial thereon). A prognostic method may also be used to determine whether a person will respond to a particular drug.

“Diagnostic method” as used herein refers to a screening procedure carried out on a subject that has previously been determined to be at risk for a particular neurodegenerative disorder due to the presentation of symptoms or the results of another (typically different) screening test.

“Functional polymorphism” as used herein refers to a change in the base pair sequence of a gene that produces a qualitative or quantitative change in the activity of the protein encoded by that gene (e.g., a change in specificity of activity; a change in level of activity). The presence of a functional polymorphism indicates that the subject is at greater risk of developing a particular disease as compared to the general population. For example, the patient carrying the functional polymorphism may be particularly susceptible to chronic exposure to environmental toxins that contribute to Alzheimer's disease. The term “functional polymorphism” includes mutations, deletions and insertions.

A “present” functional polymorphism as used herein (e.g., one that is indicative of or a risk factor for Alzheimer's disease) refers to the nucleic acid sequence corresponding to the functional polymorphism that is found less frequently in the general population relative to Alzheimer's disease as compared to the alternate nucleic acid sequence or sequences found when such functional polymorphism is said to be “absent”.

“Mutation” as used herein sometimes refers to a functional polymorphism that occurs in less than one percent of the population, and is strongly correlated to the presence of a gene (i.e., the presence of such mutation indicating a high risk of the subject being afflicted with a disease). However, “mutation” is also used herein to refer to a specific site and type of functional polymorphism, without reference to the degree of risk that particular mutation poses to an individual for a particular disease.

“Linked” as used herein refers to a region of a chromosome that is shared more frequently in family members affected by a particular disease, than expected by chance, thereby indicating that the gene or genes within the linked chromosome region contain or are associated with a marker or functional polymorphism that is correlated to the presence of, or risk of, disease. Once linkage is established, association studies (linkage disequilibrium) can be used to narrow the region of interest or to identify the risk conferring gene for Alzheimer's disease.

“Associated with” when used to refer to a marker or functional polymorphism and a particular gene means that the functional polymorphism is either within the indicated gene, or in a different physically adjacent gene on that chromosome. In general, such a physically adjacent gene is on the same chromosome and within 2 or 3 centimorgans of the named gene (i.e., within about 3 million base pairs of the named gene).

Markers (e.g., genetic markers such as restriction fragment length polymorphisms and simple sequence length polymorphisms) may be detected directly or indirectly. A marker may, for example, be detected indirectly by detecting or screening for another marker that is tightly linked (e.g., is located within two to five centimorgans) of that marker. A marker may, for example, be detected directly by a binding site.

The presence of a marker or functional polymorphism associated with a gene linked to Alzheimer's disease indicates that the subject is afflicted with Alzheimer's disease or is at risk of developing Alzheimer's disease. A subject who is “at increased risk of developing Alzheimer's disease” is one who is predisposed to the disease, has genetic susceptibility for the disease or is more likely to develop the disease than subjects in which the detected functional polymorphism is absent. While the methods described herein may be employed to screen for any type of idiopathic Alzheimer's disease, a primary application is in screening for late-onset Alzheimer's disease.

The marker or functional polymorphism may also indicate “age of onset” of Alzheimer's disease, particularly subjects at risk for Alzheimer's disease, with the presence of the marker indicating an earlier age of onset for Alzheimer's disease.

Lewy bodies are accepted as a neuropathic hallmark of Parkinson's disease and are found in up to 20% of autopsied individuals with a clinical diagnosis of what is presumed to be Alzheimer's disease. McKeith et al., Neurology 47:1113 (1996). Lewy bodies are composed of structurally altered neurofilaments and may be detected immunologically, i.e. by using immunoreactive ubiquitin stain. The precise relationships among Alzheimer's disease and dementia with Lewy bodies are not entirely clear. Clinically it has been found that subjects with dementia with Lewy bodies may also have motor symptoms of Parkinson's disease. It has been noted that Lewy-related neurites, Alzheimer's disease pathology and spongiform changes may be observed in dementia with Lewy bodies. Alzheimer's disease neuropathologic changes may also be present, including neuritic plaques and neocortical neurofibrillary tangles. See, McKeith et al., supra; Lopez et al., Neurology 54:1774 (2000). Dementia with Lewy bodies may be further divided into Diffuse Lewy Body Disease (DLBD) and Lewy Body Variant of Alzheimer's disease (LBVAD). See, e.g. Minoshima et al., Ann Neurol., 50:358 (2001), comparing cerebral metabolism among subjects with pure Alzheimer's disease, LBVAC and with pure DLBD; Hansen et al., Neurology, 40:18 (1990); McKeith et al., Neurology, 44:872 (1994).

Suitable subjects include those who have not previously been diagnosed as afflicted with Alzheimer's disease, those who have previously been determined to be at risk of developing Alzheimer's disease, and those who have been initially diagnosed as being afflicted with Alzheimer's disease where confirming information is desired. Thus, it is contemplated that the methods described herein be used in conjunction with other clinical diagnostic information known or described in the art which are used in evaluation of subjects with Alzheimer's disease or suspected to be at risk for developing such disease.

The detecting step may be carried out in accordance with known techniques (See, e.g., U.S. Pat. Nos. 6,027,896 and 5,508,167 to Roses et al.), such as by collecting a biological sample containing DNA or RNA from the subject, and then determining the presence or absence of DNA or RNA encoding or indicative of the functional polymorphism in the biological sample. Any biological sample which contains the DNA or RNA of that subject may be employed, including tissue samples and blood samples, with blood cells being a particularly convenient source.

Determining the presence or absence of DNA or RNA encoding a particular functional polymorphism may be carried out with an oligonucleotide probe labeled with a suitable detectable group, and/or by means of an amplification reaction such as a polymerase chain reaction or ligase chain reaction (the product of which amplification reaction may then be detected with a labeled oligonucleotide probe or a number of other techniques). Further, the detecting step may include the step of detecting whether the subject is heterozygous or homozygous for the particular functional polymorphism. Numerous different oligonucleotide probe assay formats are known which may be employed to carry out the present invention See, e.g., U.S. Pat. No. 4,302,204 to Wahl et al.; U.S. Pat. No. 4,358,535 to Falkow et al.; U.S. Pat. No. 4,563,419 to Ranki et al.; and U.S. Pat. No. 4,994,373 to Stavrianopoulos et al. (all U.S. Patent references cited herein be incorporated herein by reference).

Amplification of a selected, or target, nucleic acid sequence may be carried out by any suitable means. See generally, Kwoh et al., Am. Biotechnol. Lab. 8, 14-25 (1990). Examples of suitable amplification techniques include, but are not limited to, polymerase chain reaction, ligase chain reaction, strand displacement amplification (see generally G. Walker et al., Proc. Natl. Acad. Sci. USA 89, 392-396 (1992); G. Walker et al., Nucleic Acids Res. 20, 1691-1696 (1992)), transcription-based amplification (see D. Kwoh et al., Proc. Natl. Acad Sci. USA 86, 1173-1177 (1989)), self-sustained sequence replication (or “3SR”) (see J. Guatelli et al., Proc. Natl. Acad Sci. USA 87, 1874-1878 (1990)), the Qβ replicase system (see P. Lizardi et al., Bio Technology 6, 1197-1202 (1988)), nucleic acid sequence-based amplification (or “NASBA”) (see R. Lewis, Genetic Engineering News 12 (9), 1 (1992)), the repair chain reaction (or “RCR”) (see R. Lewis, supra), and boomerang DNA amplification (or “BDA”) (see R. Lewis, supra). Polymerase chain reaction is currently preferred.

Polymerase chain reaction (PCR) may be carried out in accordance with known techniques. See, e.g., U.S. Pat. Nos. 4,683,195; 4,683,202; 4,800,159; and 4,965,188. In general, PCR involves, first, treating a nucleic acid sample (e.g., in the presence of a heat stable DNA polymerase) with one oligonucleotide primer for each strand of the specific sequence to be detected under hybridizing conditions so that an extension product of each primer is synthesized which is complementary to each nucleic acid strand, with the primers sufficiently complementary to each strand of the specific sequence to hybridize therewith so that the extension product synthesized from each primer, when it is separated from its complement, can serve as a template for synthesis of the extension product of the other primer, and then treating the sample under denaturing conditions to separate the primer extension products from their templates if the sequence or sequences to be detected are present. These steps are cyclically repeated until the desired degree of amplification is obtained. Detection of the amplified sequence may be carried out by adding to the reaction product an oligonucleotide probe capable of hybridizing to the reaction product (e.g., an oligonucleotide probe of the present invention), the probe carrying a detectable label, and then detecting the label in accordance with known techniques, or by direct visualization on a gel. When PCR conditions allow for amplification of all allelic types, the types can be distinguished by hybridization with an allelic specific probe, by restriction endonuclease digestion, by electrophoresis on denaturing gradient gels, or other techniques.

DNA amplification techniques such as the foregoing can involve the use of a probe, a pair of probes, or two pairs of probes which specifically bind to DNA containing the functional polymorphism, but do not bind to DNA that does not contain the functional polymorphism. Alternatively, the probe or pair of probes could bind to DNA that both does and does not contain the functional polymorphism, but produce or amplify a product (e.g., an elongation product) in which a detectable difference may be ascertained (e.g., a shorter product, where the functional polymorphism is a deletion mutation). Such probes can be generated in accordance with standard techniques from the known sequences of DNA in or associated with a gene linked to Alzheimer's disease or from sequences which can be generated from such genes in accordance with standard techniques.

It will be appreciated that the detecting steps described herein may be carried out directly or indirectly. Other means of indirectly determining allelic type including measuring polymorphic markers that are linked to the particular functional polymorphism, as has been demonstrated for the VNTR (variable number tandem repeats) and the ApoB alleles (Decorter et al., DNA & Cell Biology 9 (6), 461-69 (1990), and collecting and determining differences in the protein encoded by a gene containing a functional variant, as described for ApoE4 in U.S. Pat. Nos. 5,508,167 and 6,027,896 to Roses et al.

Kits for determining if a subject is or was (in the case of deceased subjects) afflicted with or is or was at increased risk of developing Alzheimer's disease will include at least one reagent specific for detecting for the presence or absence of at least one functional polymorphism as described herein and instructions for observing that the subject is or was afflicted with or is or was at increased risk of developing Alzheimer's disease if at least one of the functional polymorphisms is detected. The kit may optionally include one or more nucleic acid probes for the amplification and/or detection of the functional polymorphism by any of the techniques described above, with PCR being currently preferred.

The present invention may use the variance-component procedure in SOLAR to perform genomewide scans on the quantitative trait AAO for Alzheimer's disease to map quantitative trait loci influencing AAO. The present method may be less penetrance-model dependent than the classical segregation/linkage-mapping technique, thus, it may take into account, covariate or random effects. The common regions showing evidence of linkage from independent analyses of Alzheimer's disease data sets may be further analyzed by use of the combined Alzheimer's disease data set.

Genomic screens have concentrated historically on identifying genes controlling the risk of developing a disease. However, risk is not the only important aspect of a disease. Onset of disease is also crucial, as understanding the regulation of onset could make it possible to delay onset beyond an individual's normal life span. The results as discussed below demonstrate that AAO is highly heritable and that the search for AAO genes is possible. It should be noted that AAO data are very difficult to acquire reliably, and false-negative results may be produced. With this point in mind, this result included in this application followed published standards in ascertainment for definition of reported AAO for affected individuals and reported AAE for a participant. In addition, the large sample sizes assembled in the present study for Alzheimer's disease should help to decrease the false-negative outcome.

The present genomic screen for AAO in Alzheimer's disease has identified several linkage regions for AAO, in which chromosome 12 shows the most promising results, with LOD scores >2. The APOE gene still yielded the strongest linkage effect among the newly identified regions in Alzheimer's disease, and the role of APOE in controlling onset of Alzheimer's disease was further confirmed.

The present invention is explained in greater detail in the Examples that follow. These examples are intended as illustrative of the invention and are not to be taken as limiting thereof.

Materials and Methods

### EXAMPLES

Results of a 1997 Duke University complete genomic screen identified the pericentric region of human chromsome 12 as a possible location for a gene associated with the occurrence of Alzheimer's disease. Pericak-Vance et al. JAMA 278:1237 (1997). The linkage to chromosome 12 has also been replicated in two independent sample. See, Rogaeva et al., JAMA 280:614 (1998); Wu et al., JAMA 280:619 (1998). It is known that two functional genes reside on human chromosome 12:LRP1 (Low Density Lipoprotein (LDL) receptor-related protein), located on 12 q; and A2M (a2-macroglobulin), located on 12 p. Noncoding LRP1 polymorphisms have been associated with a slightly increased risk of Alzheimer's disease in some studies, but this finding has not been replicated in all studies. See, e.g. Scott et al., Am. J. Human Genetics, 66:922 (2000). The National Institute of Mental Health AS Genetics Initiative data set showed a significant association with an insertion/deletion polymorph in the A2M Gene. Blacker et al., Nat Genet., 19:357 (1998).

The present inventors have identified evidence of loci on chromosome 12 relating to Alzheimer's disease. One of these loci segregates in families that have dementia with Lewy Body. Previously, Scott et al., supra, considered neuropathologic findings as a potential indicator of genetic heterogeneity. In the present study, families were stratified into two groups based on the presence of at least one family member with autopsy findings consistent with consensus criteria for dementia with Lewy bodies. The peak LOD of 2.18 was obtained in the eight dementia with Lewy Body families in between D12S1042 and D121090, while the remaining families generated a peak LOD of 0.58 at D12S1632 near the LRP1 locus. Simulation also determined that the increase in LOD using the dementia with Lewy Body criteria was statistically significant (p=0.035).

Six of the eight dementia with Lewy Body families are part of the DUMC LEAD family resource. These families were prioritized for re-connect and follow-up which resulted in several changes in clinical status to probably and possible Alzheimer's disease. The existing data was then reanalyzed with the new clinical statuses. This study indicated a possible peak MLOD at D12S1057 (a marker which flanks D121042 at 4cM) and is MLOD=3.01, wherein the previous MLOD=2.5. It is also important to note that of these eight families, none demonstrated evidence of linkage to either chromosome 9 or 10. Thus, the present data serves as a potential valuable resource for mapping and testing candidate loci on chromosome 12.

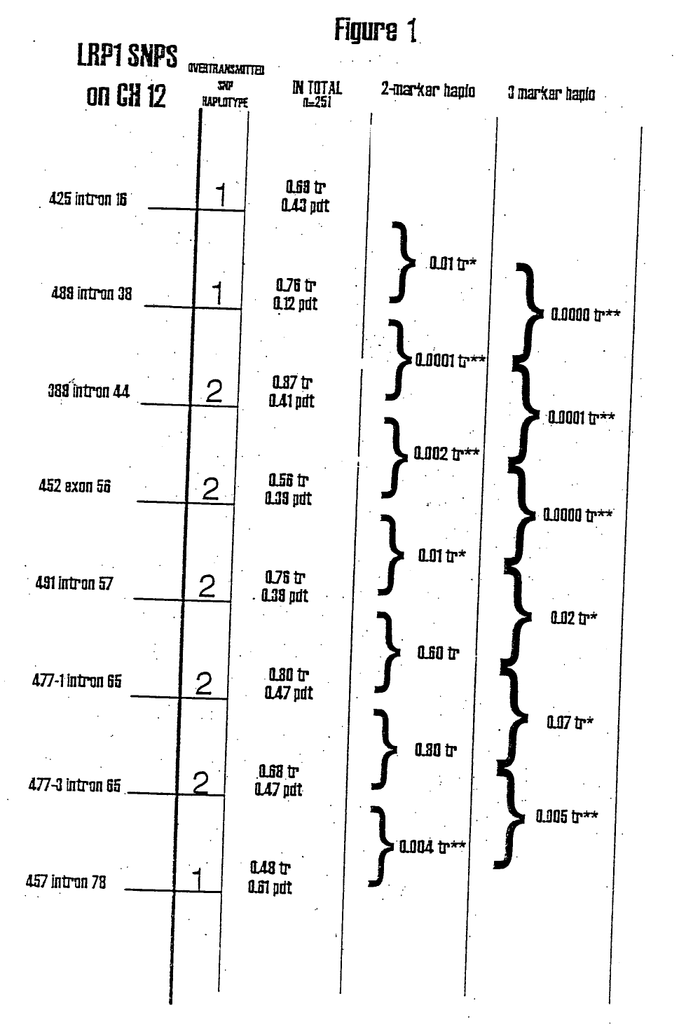

The second loci on chromosome 12 indicated Alzheimer's disease focuses on the LRP1 gene. The polymorphisms in the 5′ end of the LRP1 gene have previously been suggested as being involved in risk for Alzheimer's disease. However, the results have not been consistently replicated. Using new SNPs developed, the present invention as disclosed above, demonstrates potential evidence for association with a haplotype toward the 3′ end of the LRP1 gene. These data are illustrated in FIG. 1. Additionally, evidence suggesting an interaction between the LRP1 and the APOE locus is illustrated in this figure. This data indicates that LRP1 contributes to risk in Alzheimer's disease and that this risk may be dependent on APOE genotype.

In the specification, there has been disclosed typical preferred embodiments of the invention and, although specific terms are employed, they are used in a generic and descriptive sense only and not for purposes of limitation of the scope of the invention being set forth in the following claims.

1. A method of screening a subject for Alzheimer's disease comprising:
detecting the presence or absence of at least one or more markers linked to Alzheimer's disease, wherein the presence of said marker indicates that the subject is afflicted with or at risk of developing Alzheimer's disease, and wherein said marker is selected from the group consisting of D12S1042, D12S1090, D12S1057, D12S1632 and markers within four centimorgans thereof.

2. The method according to claim 1, wherein said marker is linked to age of onset of Alzheimer's disease.

3. The method according to claim 1, wherein said Alzheimer's disease is late-onset Alzheimer's disease.

4. The method according to claim 1, wherein said method is a diagnostic method.

5. The method according to claim 1, wherein said method is a prognostic method.

6. The method according to claim 1, wherein said subject is human.

7. A method for diagnosing a subject as having Alzheimer's disease, or as having a predisposition to Alzheimer's disease comprising:
determining the presence or absence of an allele of a polymorphic marker in the subject, wherein (i) the allele is associated with a phenotypic marker of Alzheimer's disease, and wherein (ii) the polymorphic marker is within a segment selected from the group consisting of:
a segment of chromosome 12 bordered by D12S1042 and D12S1090;
a segment of chromosome 12 within four centimorgans of D12S1042:
a segment of chromosome 12 within four centimorgans of D12S1090;
a segment of chromosome 12 within four centimorgans of D12S1057; and
a segment of chromosome 12 within four centimorgans of D12S1632.

8. The method according to claim 7, wherein said determining the presence or absence of an allele of a polymorphic marker in the subject is performed utilizing DNA or RNA.

9. The method according to claim 7, wherein said marker is linked to age of onset of Alzheimer's disease.

10. The method according to claim 7, wherein said Alzheimer's disease is late-onset Alzheimer's disease.

11-14. (canceled)

15. An oligonucleotide primer for amplification of an allele which is associated with Alzheimer's disease, wherein said allele is located at a locus in a region selected from the group consisting of:
a segment of chromosome 12 bordered by D12S1042 and D12S1090;
a segment of chromosome 12 within four centimorgans of D12S1057; and
a segment of chromosome 12 within four centimorgans of D12S1632.

16. The oligonucleotide primer of claim 15, wherein said primer is from 5 to 50 nucleotides in length.

17. The oligonucleotide primer of claim 15, wherein said allele is linked to age of onset of Alzheimer's disease.

18. The oligonucleotide primer of claim 15, wherein said Alzheimer's disease is late-onset Alzheimer's disease.

19. An assay for detecting a gene related to an age of onset disorder comprising:
providing a biological sample comprising genomic DNA from a patient suspected of having or at risk for developing said age of onset disorder;
using a probe directed toward a region of a polymorphic marker in the subject, wherein (i) the marker is associated with a phenotypic marker of Alzheimer's disease, and wherein (ii) the polymorphic marker is within a segment selected from the group consisting of:
a segment of chromosome 12 bordered by D12S1042 and D12S1090;
a segment of chromosome 12 within four centimorgans of D12S1057;and
a segment of chromosome 12 within four centimorgans of D12S1632; and
detecting duplications in the region of the genomic sequence of the group of chromosomes listed above.

20-28. (canceled)

29. A method of screening a subject for Alzheimer's disease or diagnosing a subject as having Alzheimer's disease, or as having a predisposition to Alzheimer's disease comprising:
detecting the presence or absence of at least one or more markers or an allele of a polymorphic marker linked to Alzheimer's disease, wherein the presence of said marker indicates that the subject is afflicted with, at risk of developing Alzheimer's disease or is associated with a phenotypic marker of Alzheimer's disease, and wherein said marker is selected from a marker near an LRP1 locus.

30. The method according to claim 29, wherein said marker is linked to age of onset of Alzheimer's disease.

31. The method according to claim 29, wherein said Alzheimer's disease is late-onset Alzheimer's disease.

32. The method according to claim 29, wherein said marker is located toward the 3′ end of the LRP1 gene.

33. The method according to claim 29, wherein said marker is within three centimorgans of D12S1632.

34-42. (canceled)
